(19) United States
(12) Patent Application Publication
(10) Pub. No.: US 2014/0298557 A1
(43) Pub. Date: Oct. 9, 2014

(54) PROTECTIVE SHROUD FOR A WELDING HELMET, KITS AND HELMETS INCLUDING THE SAME

(71) Applicants: Rodman Townsend, JR., Knoxville, TN (US); Eric B. Moll, Vincennes, IN (US); Ina M. Graber, Loogootee, IN (US); Mary Lou Wittmer, Montgomery, IN (US); Darlene Wittmer, Montgomery, IN (US); Mark A. Graber, Odon, IN (US); David F. Hall, Owensville, IN (US); Darren Graber, Odon, IN (US)

(72) Inventors: Rodman Townsend, JR., Knoxville, TN (US); Eric B. Moll, Vincennes, IN (US); Ina M. Graber, Loogootee, IN (US); Mary Lou Wittmer, Montgomery, IN (US); Darlene Wittmer, Montgomery, IN (US); Mark A. Graber, Odon, IN (US); David F. Hall, Owensville, IN (US); Darren Graber, Odon, IN (US)

(21) Appl. No.: 14/248,198

(22) Filed: Apr. 8, 2014

Related U.S. Application Data
(60) Provisional application No. 61/809,774, filed on Apr. 8, 2013.

(51) Int. Cl. A61F 9/06

(52) U.S. Cl. CPC A61F 9/06
USPC 2/8.2

(57) ABSTRACT

A protective shroud for a welding helmet. The shroud includes fabric that may be arranged to form an open-ended continuous loop. The loop may be sized to cover at least a portion of a user's neck. The shroud may also include one or more fasteners. Each fastener may be connected to the fabric and removably securable to the welding helmet. In other embodiments, a kit including a protective shroud, and a welding helmet including a protective shroud.

### RELATED APPLICATIONS

This application claims the benefit of U.S. Provisional Patent Application Ser. No. 61/809,774, filed Apr. 8, 2013, the entire contents of which are hereby incorporated by reference herein for all purposes.

### FIELD

The teachings herein relate to the field of welding helmets, and more particularly to protective shrouds for welding helmets and the like.

### INTRODUCTION

Welders are exposed to a number of occupational hazards including skin and eye damage from ultraviolet light, noxious fumes, and burns from spattering molten metal. Welding helmets are known to provide effective protection against photokeratitis (also known as “arc eye”) caused by exposure of a welder's eyes to intense ultra violet light emitted during welding. Some helmets include air inlet ports for providing the wearer with clean breathing air during welding.

### SUMMARY

In one embodiment, a protective shroud for a welding helmet is provided. The shroud may include fabric that is arrangeable to form an open-ended continuous loop, and one or more fasteners. The loop may be sized to cover at least a portion of a human user's neck. Each of the fastener(s) may be connected to the fabric and removably securable to the welding helmet.

In at least one embodiment, the fabric includes flame resistant material. For instance, the fabric may include a Barrweld® material made by Barrday, Inc.

In at least one embodiment, the fabric defines an upper opening, and a lower opening. The lower opening is sized to at least partially surround a user's torso, and the upper opening is sized to surround a user's head.

In at least one embodiment, each of the fastener(s) is connected to the fabric proximate the upper opening.

In at least one embodiment, the fabric includes a front chest covering portion, a rear back covering portion, and two side shoulder covering portions. The fastener(s) may include a front fastener connected to the front chest covering portion, and which are removably securable to a front face covering portion of the welding helmet, a rear fastener connected to the rear back covering portion, and which are removably securable to a back of the head covering portion of the welding helmet, and a side fastener connected to each of the two side shoulder covering portions, each side fastener being removably securable to a different lateral side of the welding helmet.

In at least one embodiment, the fabric includes a skin facing side (or inward facing side), and an opposite outward facing side. The front fastener may be connected to the outward facing side of the front chest covering portion, and the rear fastener may be connected to the skin facing side of the rear back covering portion.

In at least one embodiment, the front fastener may be securable to an interior surface of the welding helmet, and the rear fastener and side fasteners may each be securable to an exterior surface of the welding helmet.

In at least one embodiment, the fastener(s) may include one or more of loops, snaps, zippers, buttons, laces, buckles, hooks, pressure-sensitive adhesive, and magnets.

In at least one embodiment, the fabric includes a front chest covering portion, a rear back covering portion, and two side shoulder covering portions. Each of the side shoulder covering portions may include a cutout sized to permit passage of a user's arm when laterally extended.

In at least one embodiment, the fabric may include a front chest covering portion, a rear back covering portion, and two side shoulder covering portions. The rear back covering portion may include a cutout sized to at least partially surround an air inlet port of the welding helmet.

In at least one embodiment, the fabric includes a front chest covering portion, a rear back covering portion, and two side shoulder covering portions. The front chest covering portion may have opposite lateral ends. Each lateral end may be connected to one of the side shoulder covering portions. The front chest covering portion may have opposite upper and lower vertical ends, and the fastener(s) may include a front fastener connected near the upper end of the front chest covering portion, and removably securable to a front face covering portion of the welding helmet.

In at least one embodiment, the lower end of the front chest covering portion may extend below a human user's clavicle when the front fastener is secured to the front face covering portion of the welding helmet, the user is wearing the welding helmet, and the user is looking upwardly at a 45 degree angle.

In at least one embodiment, the fabric defines an upper opening sized to surround a user's head. The opening may have a perimeter and the fastener(s) may extend across at least 80% of the perimeter.

In another embodiment, a protective shroud kit for a welding helmet is provided. The kit may include a protective shroud and one or more fasteners connectable to the welding helmet, and removably securable to the one or more fasteners of the protective shroud.

In at least one embodiment, the fastener(s) include one or more of loops, snaps, zippers, buttons, laces, buckles, hooks, pressure-sensitive adhesive, and magnets.

In at least one embodiment, the fastener(s) include adhesive permanently securable to the welding helmet.

In another embodiment, a welding helmet is provided. The welding helmet includes an upper helmet, and a protective shroud. The upper helmet may include a face covering portion having a viewing window, a “back of the head” (or occiput) covering portion, and two opposed lateral side portions intermediate the face covering portion and the back of the head covering portion, each lateral side portion connecting the face covering portion to the back of the head covering portion. The face covering portion, the back of the head covering portion, and the lateral side portions may defining a cavity for receiving a user's head. The protective shroud may include fabric forming an open ended continuous loop. The fabric may define an upper opening, and a lower opening, the fabric secured to the upper helmet about substantially an entire lower perimeter of the upper helmet. The fabric may extend away from the upper helmet. When a user's head is received in the upper helmet, the protective shroud may drape over the user's body, and substantially no skin of the user may be exposed from at least the user's clavicle upwards.

### DRAWINGS

FIG. 1 is a side elevation view of a welder wearing a welding helmet;

FIG. 2 is a perspective view of the welding helmet of FIG. 1;

FIG. 3 is a perspective view of the welding helmet of FIG. 1 with protective accessories;

FIG. 4 is a perspective view of a user wearing a protective hood;

FIG. 5 is a front view of a user wearing a protective shroud in accordance with at least one embodiment;

FIG. 6 is a side view of the user wearing the protective shroud of FIG. 5;

FIG. 7 is a rear view of the user wearing the protective shroud of FIG. 5;

FIG. 8 is a front view of a user wearing a welding helmet having a protective shroud secured thereto, in accordance with at least one embodiment;

FIG. 9 is a side view of the user wearing the welding helmet of FIG. 8;

FIG. 10 is a rear view of the user wearing the welding helmet of FIG. 8;

FIG. 11 is a perspective view of a welding helmet including fasteners, in accordance with at least one embodiment;

FIG. 12 is a front interior view of a front face covering portion of the welding helmet of FIG. 12;

FIG. 13 is an enlarged partial side view of the welding helmet of FIG. 8;

FIG. 14 is a front interior view of the welding helmet of FIG. 8;

FIG. 15 is a front view of a protective shroud kit, in accordance with at least one embodiment; and

FIG. 16 is a perspective view of a user wearing a welding helmet having a protective shroud secured thereto, in accordance with at least one embodiment.

### DESCRIPTION OF VARIOUS EMBODIMENTS

As discussed above, welding helmets are known to provide effective protection against photokeratitis (also known as “arc eye”) caused by exposure of a welder's eyes to intense ultra violet light emitted during welding. Moreover, some helmets include air inlet ports for providing the wearer with clean breathing air during welding.

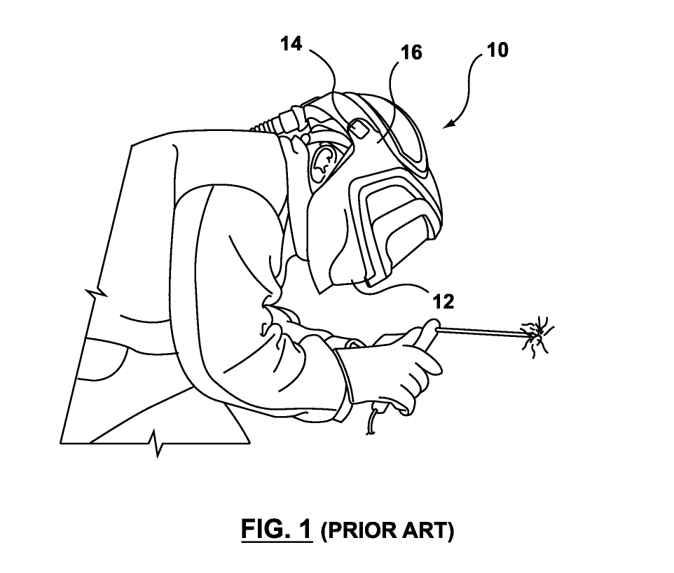

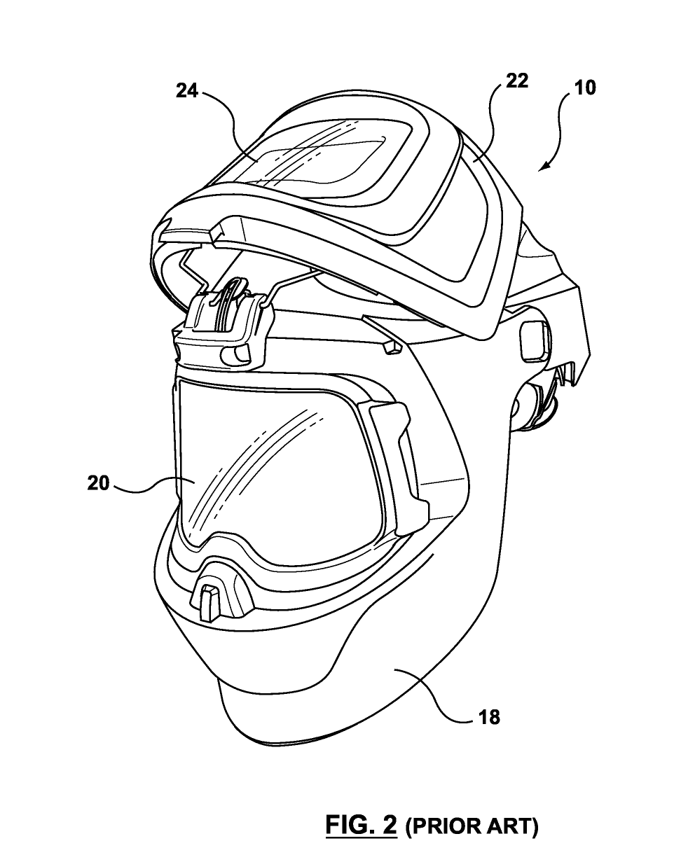

For instance, FIG. 1 shows a welder wearing a 3M® Speedglas™ welding helmet 10. As shown, welding helmet 10 includes a front face covering portion 12, a back of the head covering portion 14, and lateral side portions 16. FIG. 2 shows a perspective view of welding helmet 10. As shown, front face covering portion 12 includes a stationary portion 18. Stationary portion 18 includes a viewing window 20, and a movable portion 22 having a filtered lens 24.

Movable portion 22 can be pivoted downwardly to align the viewing window 20 and the filtered lens 24. In this position, a welder's eyes are protected from ultra violet light emitted during welding. Typically, filtered lens 24 filters out a large portion of incoming light which can make viewing through the lens 24 difficult when a bright light source, such as a welding arc, is not present. Accordingly, movable portion 22 can be pivoted upwardly to the position shown in FIG. 2 when not welding to improve visibility.

However, some welding helmets and accessories provide less than desired protection against burns, for example from molten metal that is propelled during welding.

For example, welding helmet 10 tends to provide limited protection against burns from molten metal. In particular, as shown in FIG. 1, welding helmet 10 leaves a user's ears and some other head and neck areas exposed and at risk of burns from direct contact with molten metal projecting from the user's welds or the welds of nearby welders. Furthermore, welding helmet 10 does not protect against molten metal entering welding helmet 10 under a lower edge of welding helmet 10.

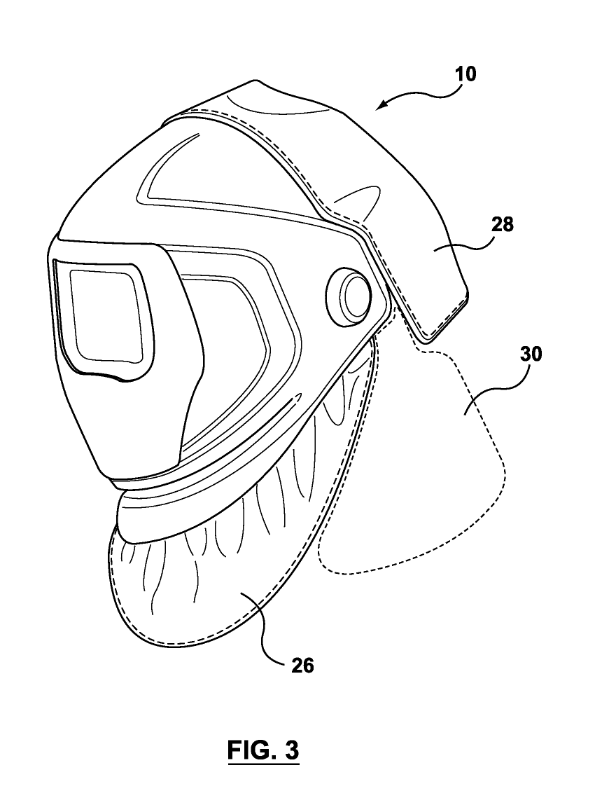

FIG. 3 shows a perspective view of welding helmet 10 fitted with a neck protector 26 and a head protector 28. Head and neck protectors 26 and 28 provide some protection against burns from spattering molten metal. However, the combination of head and neck protectors 26 and 28 still leaves an area 30 on the side of a user's neck exposed. Moreover, when a user looks upwardly, neck protector 26 tends to separate from the user's chest exposing the front of a user's neck, and when a user looks downwardly, head protector 28 moves forward exposing the back of a user's neck. Moreover, it has been observed that the head and neck protectors 26 and 28 do not prevent molten metal from entering welding helmet 10 from under an edge of the welding helmet 10 (or one of protectors 26 and 28).

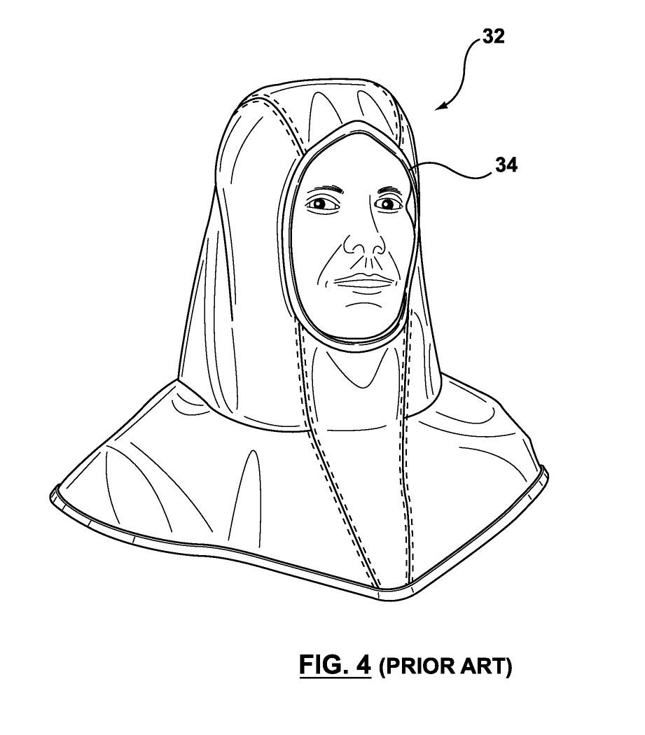

FIG. 4 shows a perspective view of a user wearing a protective hood 32, also called a balaclava-type protector. In the example shown, protective hood 32 covers a user's head, shoulder, and chest areas, and includes an opening 34 for a user's face. In use, hood 32 is put on first, followed by a welding helmet 10 on top.

However, the combination of hood 32 and welding helmet 10 still does not provide the desired level of protection to user. In particular, the combination of hood 32 and helmet 10 tends to leave the user's face exposed to molten metal that can enter from under a lower edge of welding helmet 10. Furthermore, when a user is working in an environment where molten metal is present behind the user (i.e., when a number of welders are operating in the same space), such molten metal can enter any gaps between the helmet 10 and the hood 32, which can lead to the molten metal damaging the hood 32 and is some cases burning or causing other injury to the user wearing the hood 32. Moreover, in the example shown, protective hood 32 is made of a blend of cotton and polyester that may melt, drip and/or burn if exposed to hot molten metal.

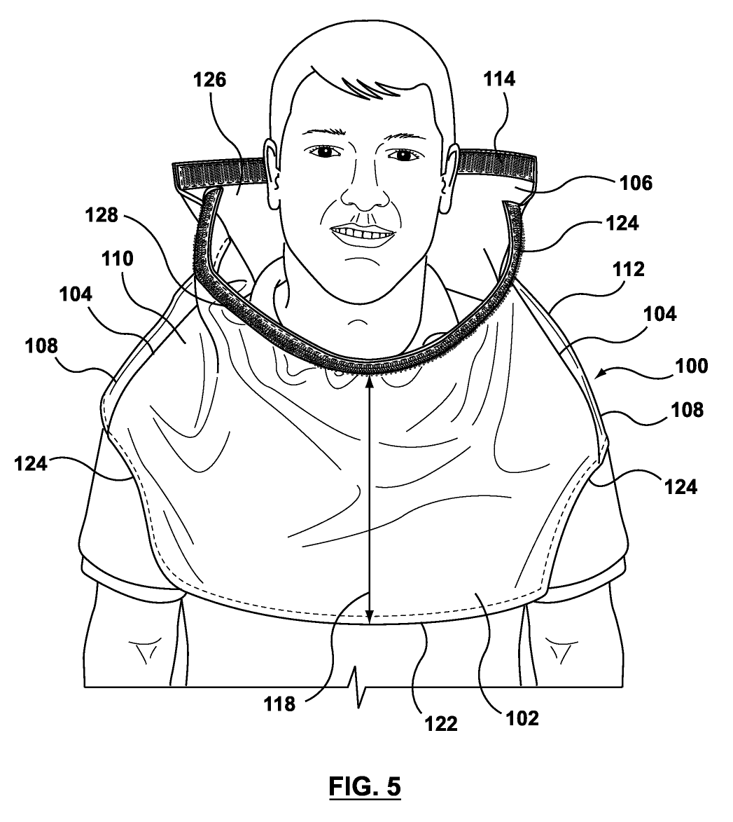

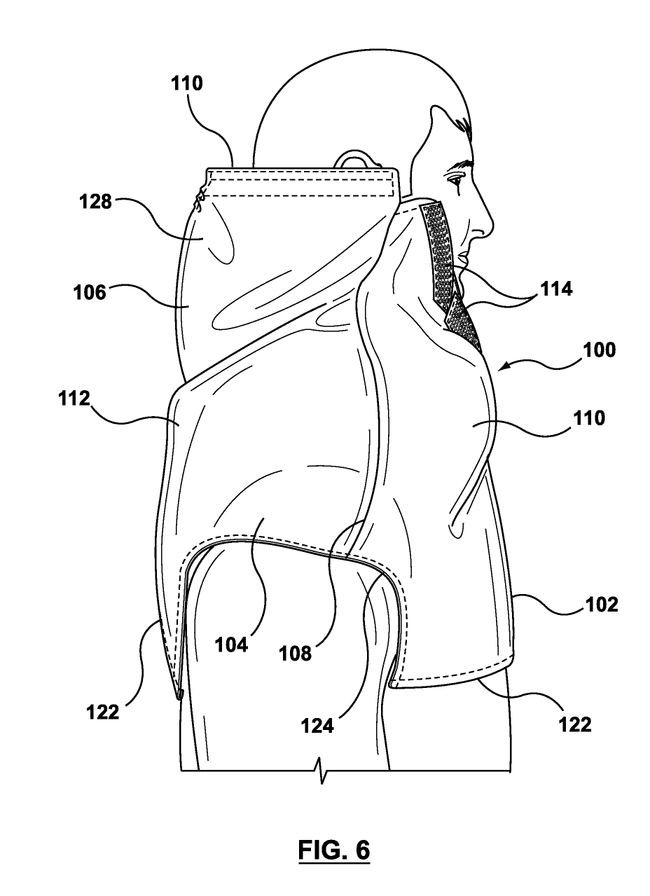

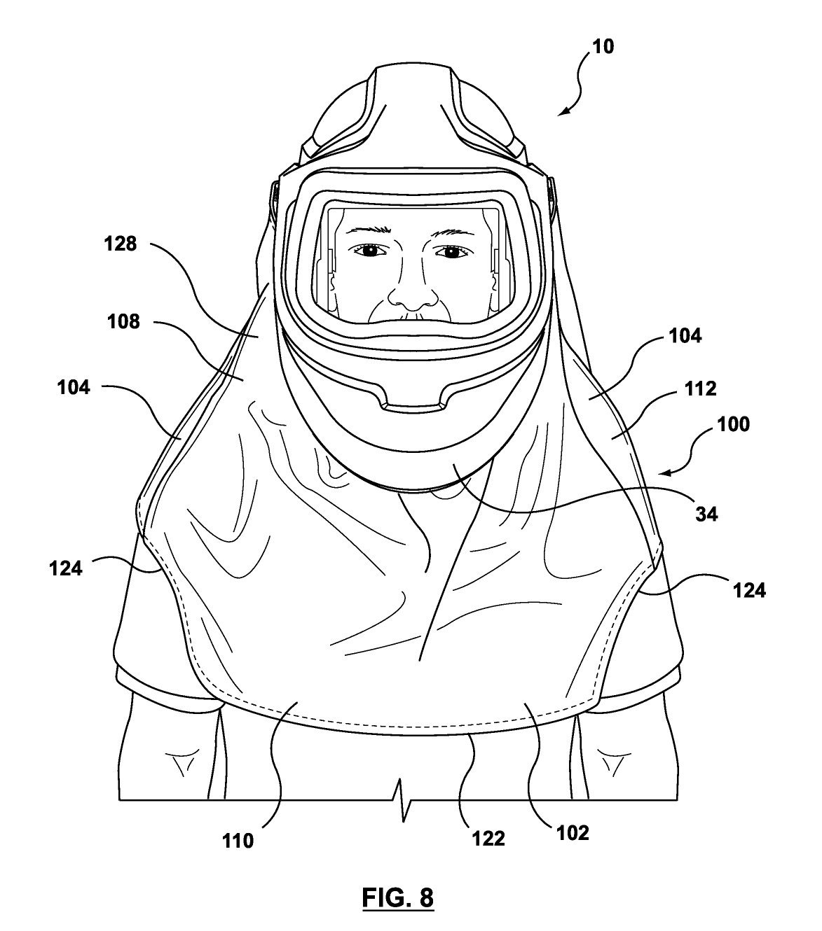

Reference is now made to FIGS. 5-10, which show a protective shroud according to some of the teachings herein. In particular, FIGS. 5, 6, and 7 show a front, side and rear view, respectively, of a user wearing a protective shroud 100, in accordance with at least one embodiment. FIGS. 8, 9, and 10 show a front, side and rear view, respectively, of a user wearing a protective shroud 100 secured to a welding helmet 10. In the example shown, shroud 100 is a fabric covering that can be draped over the upper body of a user. As shown, shroud 100 includes a front chest covering portion 102, a rear back covering portion 104, and two side shoulder covering portions 104 intermediate the front and rear portions 102 and 104.

Shroud 100 may include one or more pieces of fabric assembled to surround at least a portion of the neck of a human user, and in some embodiments at least the entire neck of the user. In the example shown, shroud 100 includes a front and rear fabric portions 110, and 112 connected at stitched seams 108 which extend through side portions 104. In at least one alternative embodiment, shroud 100 includes one fabric portion or a plurality of connected or connectable fabric portions, which can form a contiguous loop sized to cover at least a user's neck.

In some examples, two or more fabric portions are permanently connected together by stitches, staples, adhesive, or welds, for example.

In some examples, two or more fabric portions are removably connected together by hooks, loops, snaps, zippers, buttons, laces, buckles, pressure-sensitive adhesive, or magnets. For example, one or more of stitched seams 108 of shroud 100 may be substituted by one or more of the aforementioned removable connections to provide a shroud that can be opened on at least one side. In some cases, this may permit a better fit about, e.g. the neck of a user. For example, this may permit a diameter of a neck encircling portion to be narrower than a user's head, which may otherwise make sliding shroud 100 on over a user's head difficult if the shroud 100 could not be opened.

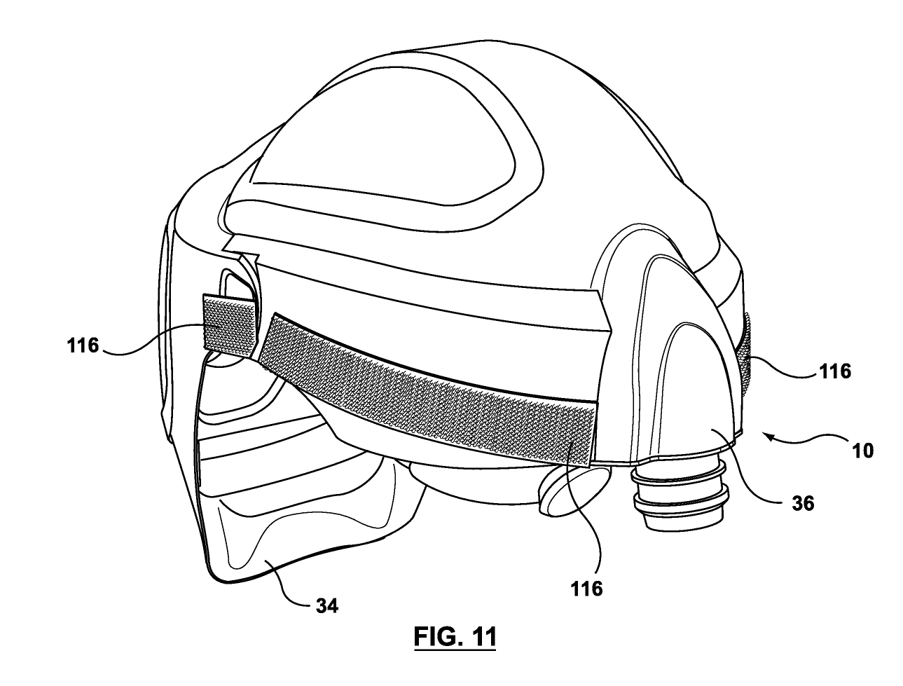

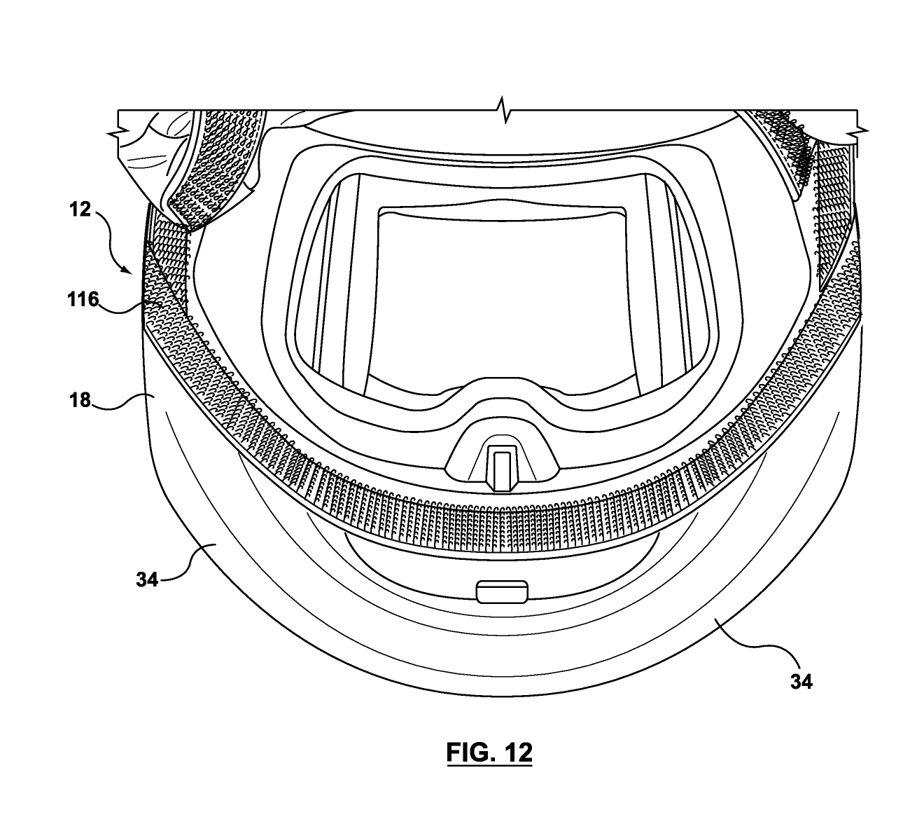

Shroud 100 as shown defines an upper opening 110 sized and shaped to surround a user's head, and a lower opening 112 sized to surround a user's torso. As shown, shroud 100 includes a plurality of fasteners 114 connected to the front and rear fabric portions 110 and 112 proximate the upper opening 110. In the example shown, fasteners 114 are lengths of hook and loop fasteners (e.g. Velcro®) that are removably securable to mating hook fasteners 116 connected to helmet 10 (see FIGS. 11 and 12). In alternative embodiments, fasteners 114, and 116 may include one or more of mating snaps, zippers, buttons, laces, buckles, pressure-sensitive adhesive, or magnets.

As shown in FIGS. 8 to 10, shroud 100 generally provides total or “360°” coverage against skin exposure between helmet 10 and the upper torso of the user. In particular, shroud 100 is shown secured to substantially the entire perimeter of helmet 10. For example, fasteners 116 connected to welding helmet 10 may extend across up to 50% or more (or even up to 80% or more) of the perimeter of welding helmet 10. This may provide protection to the user's ears, and neck areas that may be left exposed when wearing welding helmet 10 alone or in combination with known accessories (for comparative purposes see FIGS. 1 and 3).

In the example shown, shroud 100 extends downwardly to cover not only the user's neck area, but also portions of the user's chest, shoulders and back. In at least one embodiment, a height 118 of front chest covering portion 102 is selected to permit a human user to look upwardly (e.g. to a weld above eye level) without exposing skin. For example, the collar of many shirts provides a neck opening not extending below a wearer's clavicle. Accordingly, in at least one embodiment, length 118 is selected so that chest covering portion 102 extends below a user's clavicle when the user is looking upwardly (e.g. at a 45° angle). A height 118 that provides this feature may depend upon a size of the user, and the location of fasteners 114 and 116. Generally, height 118 may be greater for a larger user, and the farther the location of fasteners 116 from a lower edge 34 of front face covering portion 12. In some cases, height 118 may be selected to extend below a user's xiphoid process.

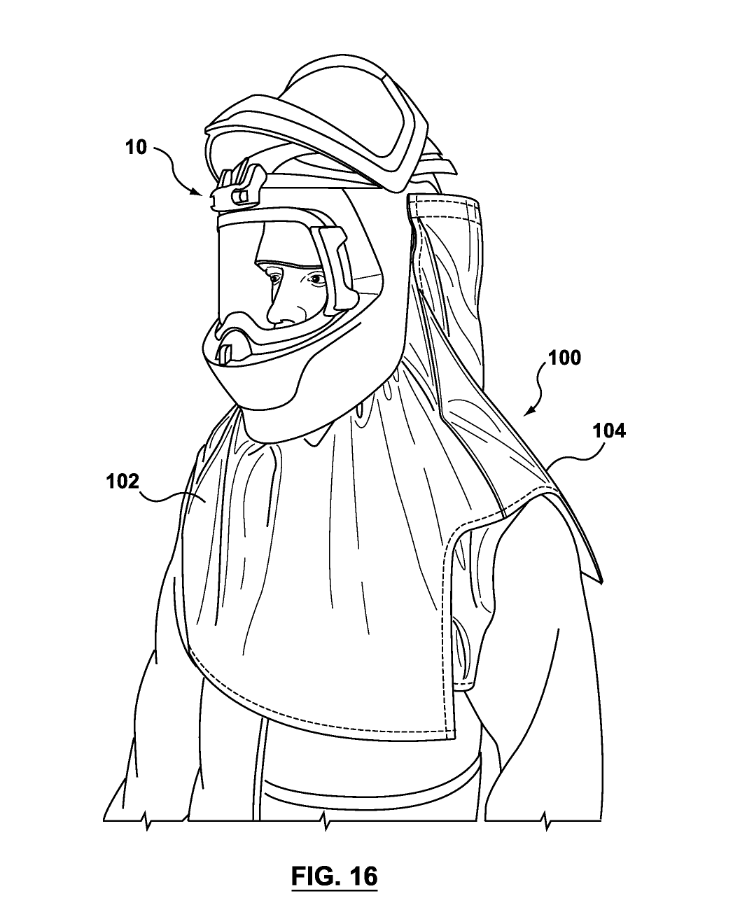

In some embodiments, the sizing of the shroud 100 can be based on a “one-size fits all” approach, for example a shroud 100 sized and shaped to be operable for up to a 95th percentile male, or a 95th percentile female. In some embodiments, the sizing of the shroud 100 can be adjusted or customized for users of different shapes and sizes (for example the shroud may be offered in a small, medium, large, extra-large, and XXL sizes, and/or in a tall or long model). For example, FIG. 16 shows a helmet 10 including a shroud 100 having front chest covering and rear back covering portions 102 and 104 that are lengthened as compared with the examples illustrated in FIGS. 5 to 10.

Shroud 100 as shown is made of a flame resistant material sold by Barrday Inc. under the name Barrweld®. Barrweld is fabric made of 40% Twaron® (aramid) and 60% Lenzing FR® Rayon which is tightly woven to protect against weld spatter and to resist pin holes. In alternative embodiments, shroud 100 is made of any one or more suitable flame resistant materials including but not limited to Barrweld, synthetic or natural leathers, Kevlar, Nomex®, and M5 fiber for example. In at least one embodiment, shroud 100 is made of TenCate TuffWeld® which is a fabric made of 60% Lenzing FR Rayon, and 40% Kevlar (aramid).

In at least one embodiment, shroud 100 resists separating from a human user's torso during use. This may help to prevent molten metal from getting under a lower edge of shroud 100 and burning the wearer's clothing or skin. In at least one example, shroud 100 has sufficient weight and flexibility to drape downwardly when the human user is looking upwardly (e.g. at a 45° angle toward a weld above eye level). In contrast, neck protector 26 shown in FIG. 3 has a more rigid structure which moves and lifts away from the human user's body when the human user angles their head with helmet 10 to look upwardly.

In some cases, a weighted trim (e.g. small pieces or a length of heavy material such as metal) may be incorporated (e.g. sown in) to shroud 100 (e.g. proximate lower edge 122). This may help shroud 100 to resist lifting from a wearer's body during use. In some cases, front chest covering portion 102 may be connected or connectable to rear back covering portion 104 from under a human user's arms. For example, a strap, string or length of fabric (not shown) may extend under each of a human user's arms to connect the front chest covering portion 102 directly to the rear back covering portion 104. This may hold the front chest covering portion 102 within a proximity to the rear back covering portion 104 and thereby limit the movement of the front chest covering portion 102 away from the human user's torso. In some examples, the front chest covering portion 102 is releasably connected to the back covering portion 104 from under the human user's arms. For example, the front chest covering portion 102 and the rear back covering portion 104 may be directly releasably connected using one or more of loops, hooks, snaps, zippers, buttons, laces, buckles, pressure-sensitive adhesive, and magnets.

In at least one embodiment, one or more of front chest covering portion 102, and rear covering portion 104 may be releasably connectable to a human user's clothing. For example, the human user's clothing may connect to one or both of front chest covering portion 102, and rear back covering portion 104 using one or more of loops, hooks, snaps, zippers, buttons, laces, buckles, pressure-sensitive adhesive, and magnets. In some cases, this may hold one or both of front chest covering portion 102 and rear back covering portion 104 against the human user's clothing to help prevent molten metal from spattering under lower edge 122 of shroud 100 and burning a human user's clothing or skin.

Each side shoulder covering portion 106 as shown includes a cutout 124. Each cutout 124 may provide a human user with enhanced shoulder and arm mobility. For example, cutout 124 may provide a passage for a human user to extend their arm laterally or forwardly. This may reduce the resistance of shroud 100 to upward and outward arm movements. Furthermore, cutouts 124 may help reduce shoulder strain caused by supporting the weight of fabric displaced during upward and outward arm movements. Additionally, by providing a passage for the movement of the human user's arms, cutout 124 may limit the displacement of front chest covering portion 102 away from a human user's torso when the human user moves their arms upwardly (e.g. to weld objects above eye-level). This may help to prevent widening openings under lower edge 122 through which molten metal may enter a human user's helmet 10. In at least one embodiment, one or both side shoulder covering portions 106 does not include a cutout 124.

Shroud 100 as shown includes fasteners 114 for removably securing to welding helmet 10. As shown, shroud 100 includes a skin facing (or inward facing) side 126, and an opposite outward facing side 128. Some fasteners 114 are shown connected to skin facing side 126, and some fasteners 114 are shown connected to outward facing side 128. More specifically, in the example shown, some of fasteners 114 (“front fasteners”) are connected to the outward side of front chest covering portion 102, some of fasteners 114 (“rear fasteners”) are connected to the skin facing side of rear back covering portion 104, and some of fasteners 114 (“side fasteners”) are connected to both the skin facing side and outward facing side of side shoulder covering portions 106. In alternate embodiment, each of the front, side and rear fasteners are connected to one or both of skin facing and outward facing sides 126 and 128 of shroud 100.

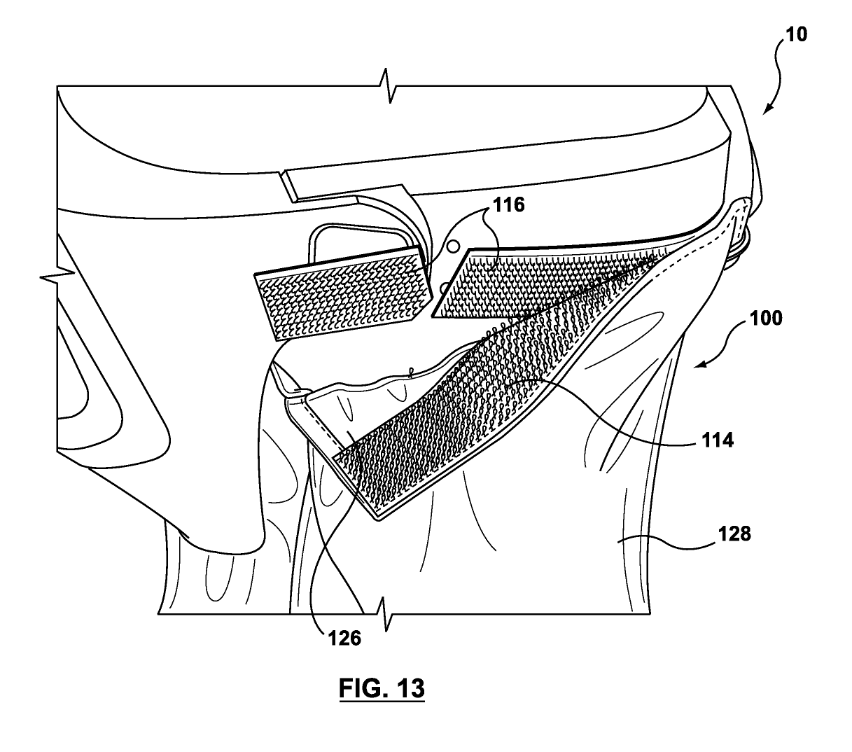

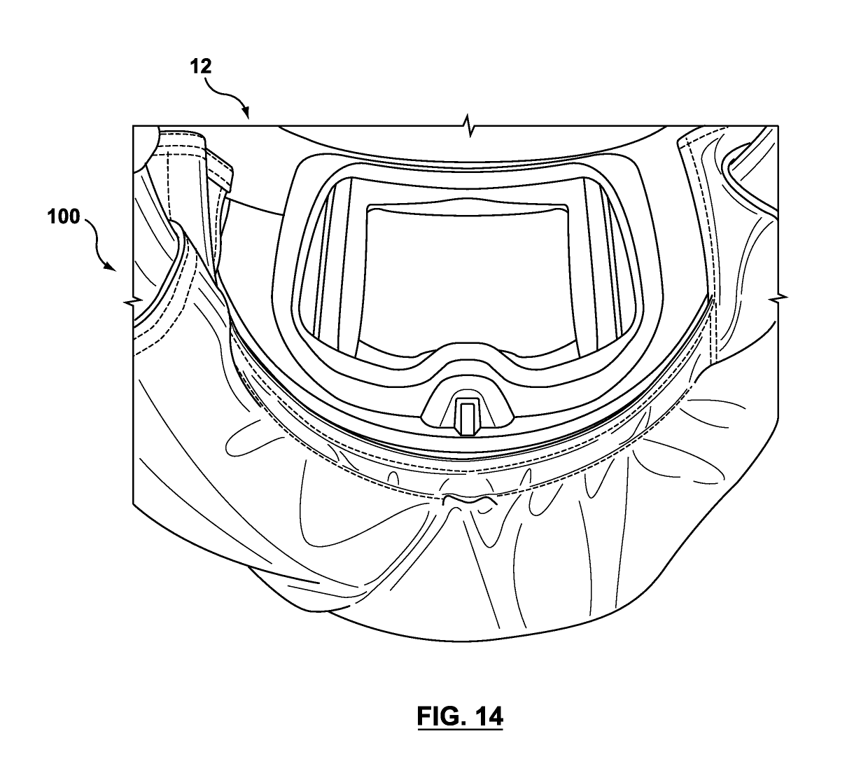

Reference is now made to FIGS. 11 to 14. FIGS. 11 and 13 show a perspective view of an exterior of welding helmet 10 having fasteners 116 connected thereto, with and without shroud 100 secured respectively, in accordance with at least one embodiment. FIGS. 12 and 14 show a perspective view of an interior of front face covering portion 10 of welding helmet 10 having fasteners 116 connected thereto, with and without shroud 100 secured respectively, in accordance with at least one embodiment. In the example shown, fasteners 114 connected to the skin facing side 126 of shroud 100 are positioned to align with fasteners 116 connected an exterior surface of welding helmet 10. Similarly, fasteners 114 connected to the outward facing side 128 of shroud 100 are shown positioned to align with fasteners 116 connected to an interior surface of welding helmet 10.

For example, in FIGS. 11 and 13, there are fasteners 116 shown connected to an exterior of the back of the head covering portion 14, and lateral side portions 16, which are positioned to align with fasteners 114 connected to the skin facing side of rear back covering portion 104 and side shoulder covering portion 106. Similarly, in FIGS. 12 and 14, there are fasteners 116 shown connected to an interior stationary portion 18 of front face covering portion 12, which are positioned to align with fasteners 114 connected to the outward facing side of front chest covering portion 102.

In at least one embodiment, securing front chest covering portion 102 to an interior of front face covering portion 12 may assist with keeping the front chest covering portion 102 closer to the human user's torso. For example, when the human user tilts their head, along with welding helmet 10, upwardly, front chest covering portion 102 may continue to extend directly downwardly from the point of connection between fasteners 114, and 116. In contrast, if front chest covering portion 102 is secured to an exterior of front face covering portion 12, then when the human user tilts their head, along with welding helmet 10, upwardly, lower edge 34 may urge front chest covering portion 10 away from the human user's torso. Still, in at least one embodiment, front chest covering portion 102 is secured to an exterior of front face covering portion 12.

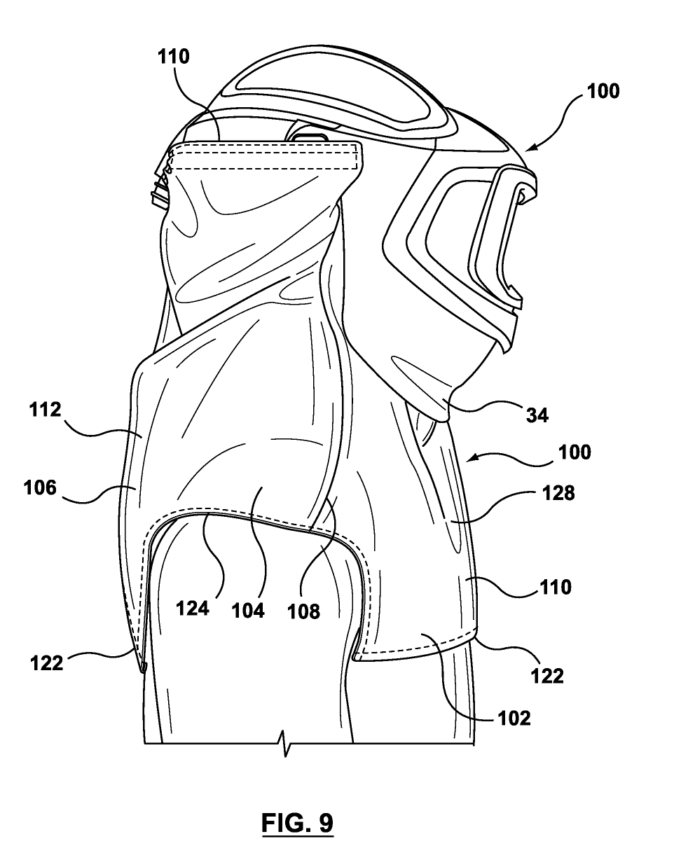

In at least one embodiment, securing rear back covering portion 104 of shroud 100 to an exterior of back of the head covering portion 14 (as shown in FIGS. 9, 10 and 13) may improve the comfort of welding helmet 10. Whereas some of the lower interior of face covering portion 12 may be spaced from the human user by design and provide an unobtrusive location to secure shroud 100, the lower interior of back of the head covering portion 14 may be designed to abut the human user's head. Accordingly, securing rear back covering portion 104 to an exterior of back of the head covering portion 14 may avoid uncomfortably interfering with the fit of welding helmet 10. Still, in at least one embodiment, rear back covering portion 104 is secured to an interior of back of the head covering portion 14.

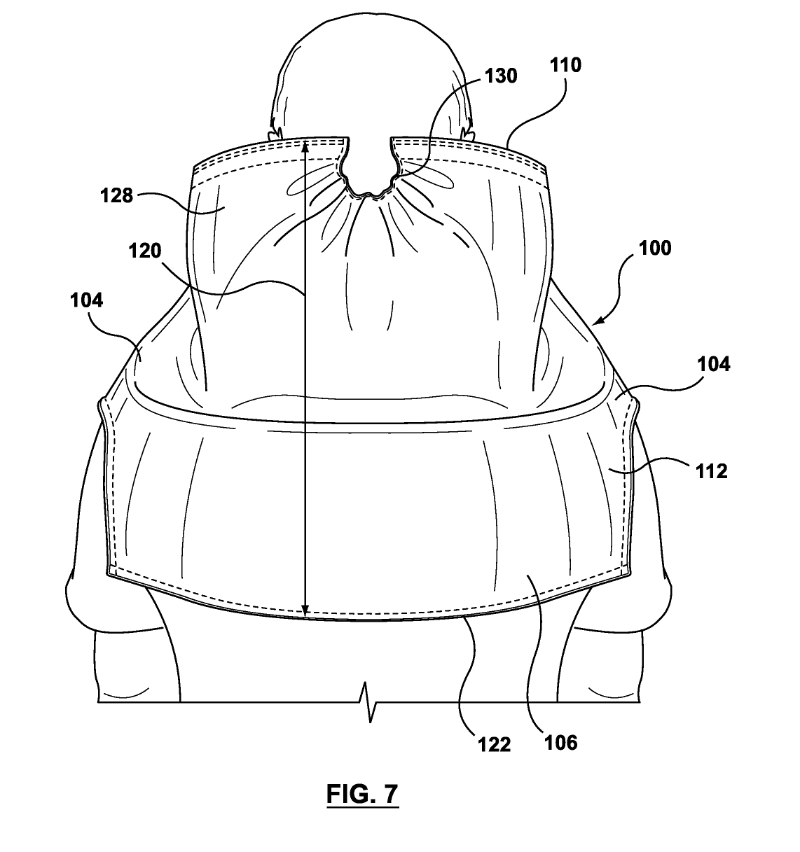

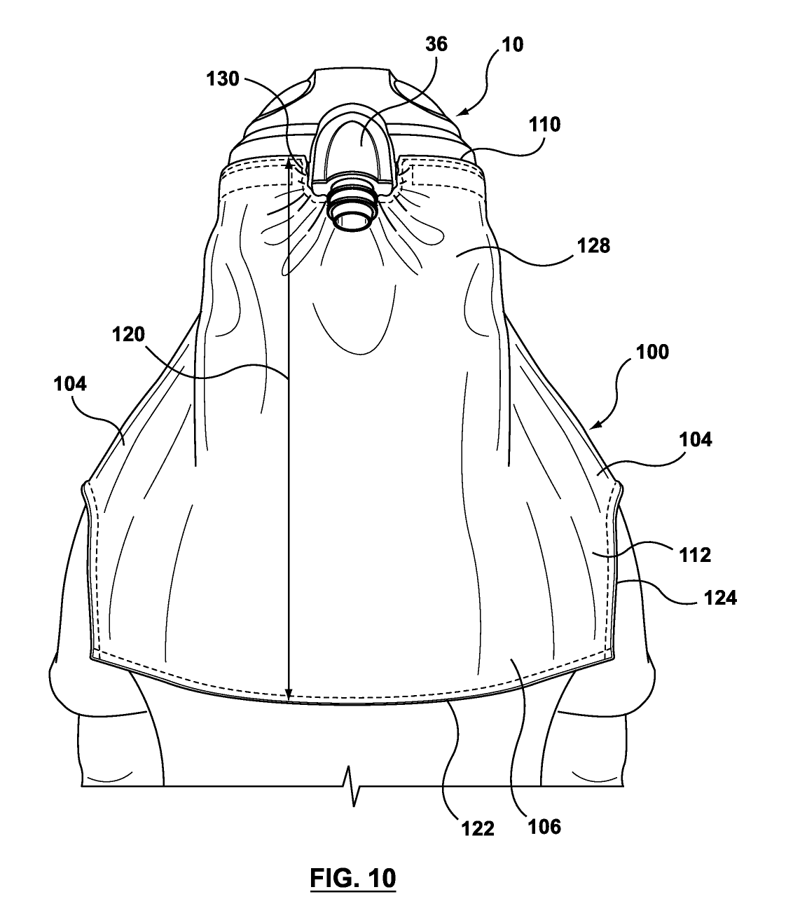

Referring now to FIGS. 7 and 10, shroud 100 is shown including a cutout 130 sized to receive an air inlet port 36 of welding helmet 10. In at least one embodiment, cutout 130 is positioned to align with and at least partially surround air inlet port 36 when shroud 100 is secured to welding helmet 10. This may permit an air hose (not shown) to be connected to air inlet port 36 while shroud 100 is secured to welding helmet 10. This may also reduce openings or spaces between shroud 100 and welding helmet 10 in the vicinity of air inlet port 36 where molten metal can enter.

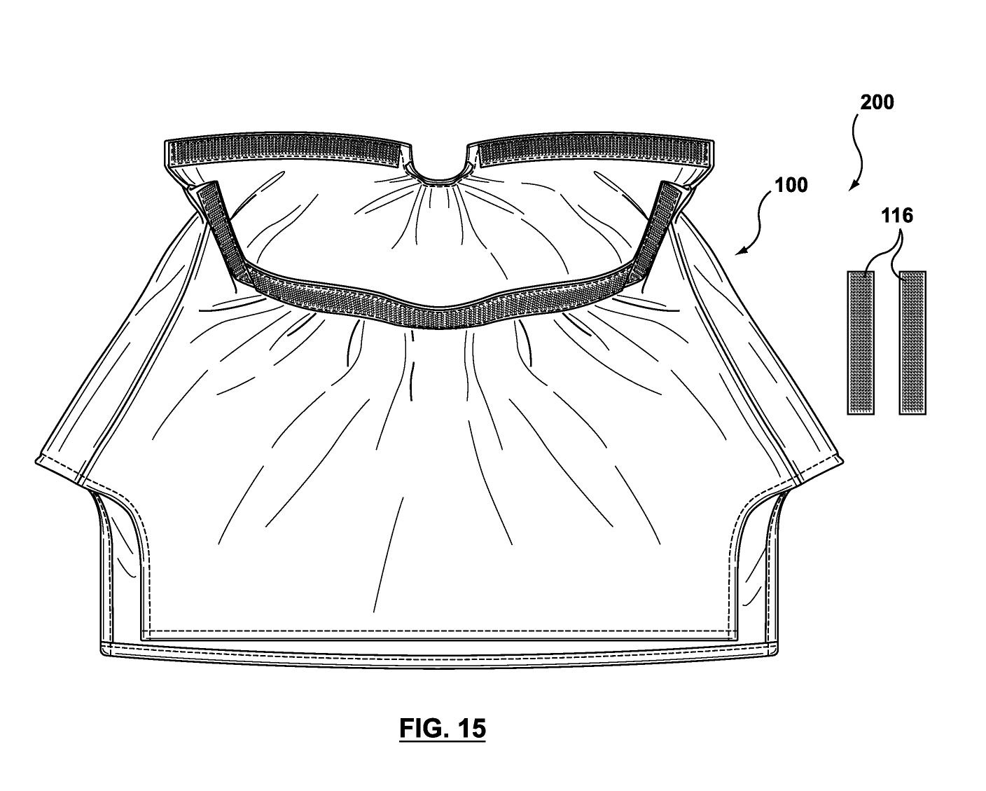

FIG. 15 shows a front view of protective shroud kit 200, in accordance with at least one embodiment. Protective shroud kit 200 is shown including a protective shroud 100, and a plurality of fasteners 116. Fasteners 116 may be later connected to a welding helmet (e.g. welding helmet 10) to retrofit the welding helmet to be removably securable to protective shroud 100. For example, fasteners 116 or protective shroud kit 200 more generally, may include, e.g. adhesive, clamps, hooks, or magnets for connecting fasteners 116 to a welding helmet. In at least one embodiment, fasteners 116 include an adhesive backing.

Although in at least some of the aforementioned embodiments shroud 100 is removably securable to a welding helmet 10, in alternative embodiments a welding helmet includes a permanently secured shroud 100. The shroud 100 may be permanently secured to the welding helmet by welds, screws, adhesive, or by clamping shroud 100 between portions of the welding helmet for example.

1. A protective shroud for use with a welding helmet, comprising:
fabric forming an open ended continuous loop, the fabric defining an upper opening, and a lower opening, the fabric securable to an upper helmet of the welding helmet about substantially an entire lower perimeter of the upper helmet, the fabric extending away from the upper helmet,
wherein when a user's head is received in the upper helmet, the protective shroud drapes over the user's body, and at least substantially no skin is exposed from at least the user's clavicle upwards;
wherein the fabric includes flame resistant material.

2. A protective shroud for a welding helmet, the shroud comprising:
fabric arrangeable to form an open-ended continuous loop, the loop sized to cover at least a portion of a user's neck; and
one or more fasteners, each connected to the fabric and removably securable to the welding helmet.

3. The protective shroud of claim 2, wherein the fabric includes flame resistant material.

4. The protective shroud of claim 3, wherein the fabric includes Barrweld material.

5. The protective shroud of claim 2, wherein:
the fabric defines an upper opening, and a lower opening,
the lower opening is sized and shaped to surround a user's torso, and
the upper opening is sized and shaped to surround a user's head.

6. The protective shroud of claim 5, wherein each of the fastener(s) is connected to the fabric proximate the upper opening.

7. The protective shroud of claim 2, wherein:
the fabric includes a front chest covering portion, a rear back covering portion, and two side shoulder covering portions,
the fastener(s) including
a front fastener connected to the front chest covering portion, and removably securable to a front face covering portion of the welding helmet,
a rear fastener connected to the rear back covering portion, and removably securable to a back of the head covering portion of the welding helmet, and
a side fastener connected to each of the two side shoulder covering portions, each side fastener removably securable to a different lateral side of the welding helmet.

8. The protective shroud of claim 7, wherein:
the fabric includes a skin facing side, and an opposite outward facing side,
the front fastener is connected to the outward facing side of the front chest covering portion, and
the rear fastener is connected to the skin facing side of the rear back covering portion.

9. The protective shroud of claim 7, wherein:
the front fastener is securable to an interior surface of the welding helmet; and
the rear fastener and side fasteners are each securable to an exterior surface of the welding helmet.

10. The protective shroud of claim 2, wherein the fastener(s) include one or more of loops, snaps, zippers, buttons, laces, buckles, hooks, pressure-sensitive adhesive, and magnets.

11. The protective shroud of claim 2, wherein:
the fabric includes a front chest covering portion, a rear back covering portion, and two side shoulder covering portions, and
each of the side shoulder covering portions includes a cutout sized to permit passage of a user's arm when laterally extended.

12. The protective shroud of claim 2, wherein:
the fabric includes a front chest covering portion, a rear back covering portion, and two side shoulder covering portions, and
the rear back covering portion includes a cutout sized to at least partially surround an air inlet port of the welding helmet.

13. The protective shroud of claim 2, wherein:
the fabric includes a front chest covering portion, a rear back covering portion, and two side shoulder covering portions,
the front chest covering portion has opposite lateral ends, each lateral end connected to one of the side shoulder covering portions,
the front chest covering portion having opposite upper and lower vertical ends, and
the fastener(s) include a front fastener connected proximate the upper end of the front chest covering portion, and removably securable to a front face covering portion of the welding helmet.

14. The protective shroud of claim 13, wherein:
the lower end of the front chest covering portion extends below a user's clavicle when the front fastener is secured to the front face covering portion of the welding helmet, the user is wearing the welding helmet, and the user is looking upwardly at a 45 degree angle.

15. The protective shroud of claim 2, wherein:
the fabric defines an upper opening sized to surround a user's head, the opening having a perimeter; and
the fastener(s) extend across at least 80% of the perimeter.

16. A protective shroud kit for a welding helmet, the kit comprising:
the protective shroud of claim 2; and
one or more second fasteners connectable to the welding helmet, and removably securable to the one or more fasteners of the protective shroud.

17. The kit of claim 16, wherein the second fastener(s) include one or more of loops, snaps, zippers, buttons, laces, buckles, hooks, pressure-sensitive adhesive, and magnets.

18. The kit of claim 16, wherein the second fastener(s) include adhesive permanently securable to the welding helmet.

19. A welding helmet comprising:
an upper helmet including
a face covering portion having a viewing window,
an back of the head covering portion, and
two opposed lateral side portions intermediate the face covering portion and the back of the head covering portion, each lateral side portion connecting the face covering portion to the back of the head covering portion,
the face covering portion, the back of the head covering portion, and the lateral side portions defining a cavity for receiving a user's head; and
a protective shroud including
fabric forming an open ended continuous loop,
the fabric defining an upper opening, and a lower opening,
the fabric secured to the upper helmet about substantially an entire lower perimeter of the upper helmet,
the fabric extending away from the upper helmet,
when a user's head is received in the upper helmet, the protective shroud drapes over the user's body, and at least substantially no skin is exposed from at least the user's clavicle upwards.

20. The welding helmet of claim 19, wherein the fabric includes flame resistant material.
